(19) United States
(12) Patent Application Publication
(10) Pub. No.: US 2003/0027148 A1
(43) Pub. Date: Feb. 6, 2003

(54) Method and device for assaying molecules using RF integrated passive components

(76) Inventors: Allyson Beuhler, Downers Groves, IL (US); Irina Shmagin, St. Itasca, IL (US); Lawrence Lach, Chicago, IL (US)

Correspondence Address: Frank C. Nicholas, CARDINAL LAW GROUP, Suite 2000, 1603 OrringtonAvenue, Evanston, IL, 60201, US

(21) Appl. No.: 09/916,108

(22) Filed: Jul. 26, 2001

(51) Int. Cl. C12Q 1/68; C12M 1/34; H04B 1/40

(52) U.S. Cl. 435/6; 435/287.2; 455/74

(57) ABSTRACT

A method and device of detecting a molecule, such as DNA or RNA, is provided. The device includes a substrate having an array of at least one biodetection site. The biodetection site is in contact with an electrical resonator. A biochemical probe is applied to the site and is subsequently interacted with the target biomolecule at the site. The magnetic properties of the biodetection site are measured and may be correlated with the presence and quantity of biomolecules at the site. Methods of using the device to detect the presence and determine the quantity of molecules at the site are also provided.

### CROSS-REFERENCE TO RELATED APPLICATIONS

This application claims the benefit of co-pending U.S. patent application Ser. No. 09/612,792, filed Jul. 10, 2000, to inventors Beuhler et al. (Attorney Docket No. CMO13991), herein incorporated by reference in its entirety.

### FIELD OF THE INVENTION

This invention relates generally to systems for assaying target unknown molecules, in particular biologically significant molecules (“biomolecules”). More particularly, this invention relates to devices that use RF integrated passive components to detect the presence of and assay the quantity of biomolecules at a test site.

### BACKGROUND OF THE INVENTION

It is often desirable to determine the presence of a given biomolecule in a biological sample. For example, it is useful to know whether a particular type of DNA is present in samples used for genetic diagnosis and research or for disease diagnosis and research. It is also desirable to know if a particular antibody is present in samples used for toxicology testing. It is further desirable to know if a particular molecule is present in samples used for agricultural and pharmaceutical development. Assays for biomolecules typically detect ligands such as cells, antibodies and anti-antibodies. Ligands may be defined as molecules that are recognized by a particular receptor. Ligands may include, without limitation, agonists and antagonists for cell membrane receptors, toxins, venoms, oligo-saccharides, proteins, bacteria, and monoclonal antibodies.

As one example, in a typical method for detecting DNA, the sample target DNA is tagged with a fluorescent moiety. Using techniques known to those skilled in the art, the sample DNA is washed over the substrate and allowed to hybridize to the oligonucleotides on the substrate. If the sample contains DNA that is complementary to an oligonucleotide on the substrate, the DNA will bind to its complementary strand. When the sample and substrate are exposed to laser light, the site where DNA has bound the oligonucleotide will fluoresce and the presence of the DNA can then be detected.

In another example, an antibody or protein can be bound to a solid support. The complementary protein or antibody to be detected is tagged with a fluorescent moiety and washed over the substrate. The complementary protein or antibody binds to its complement on the surface and then can be detected with a fluorescent reader or laser scanner.

There are several disadvantages to using fluorescence assays to detect DNA or other biomolecules. One disadvantage is that these assays generally have low sensitivity, which requires a high density of labeled oligonucleotides or other biomolecule to obtain a strong signal. This is a particular disadvantage when the sample size is very small or there are very few complementary oligonucleotides in the sample. Another disadvantage is that highly specialized equipment, such as a fluorescent scanner, is required to detect the fluorescent tag of the DNA. Also, there is very little standardization in existing assays.

A number of techniques have been developed for molecular structure detection. In DNA and RNA sequence detection, two procedures are generally used, autoradiography and optical detection.

The use of any radioactive label is associated with several disadvantages. First, prolonged exposure to radioactive elements increases the risk of acquiring genetic diseases, such as cancer. As such, precautions must be implemented when using radioactive markers or labels to reduce the exposure to radioactivity. Typically, workers must wear a device to continually monitor radioactive exposure. In addition, pregnant females should take additional precautions to prevent the occurrence of genetic mutations in the unborn.

Further, the incorporation of a radioactive label into a nucleic sequence increases the complexity and cost of the entire sequence analysis process. Although the chemistry is commonplace, it nonetheless necessitates an additional step.

Optical detection of fluorescent labeled receptors has also been utilized to detect molecular binding. Briefly, for DNA sequence analysis applications, a base-specific fluorescent dye is attached covalently to the oligonucleotide primers or to the chain terminating dideoxynucleotides. The approximate absorption wavelength for each dye is chosen and used to excite the dye. If the absorption spectra of the dyes are close to each other, a specific wavelength can be chosen to excite the entire set of dyes.

A separate optical detection technique involves the use of a dye, for example, ethidium bromide, which stains duplexed nucleic acids. The fluorescence of these dyes exhibits an approximate 20-fold increase when it is bound to duplexed DNA or RNA, when compared to the fluorescence exhibited by unbound dye, or dye bound to single-stranded DNA. This type of dye is used to detect the presence of hybridized DNA (or RNA) during a hybridization experiment.

The various dyes employed in using fluorescent labeled molecules have been shown to be mutagenic and, hence, carcinogenic. Additionally, the use of multiple fluorescent dyes may affect the electrophoretic mobility of the labeled molecules. Furthermore, large and expensive equipment must be purchased for the operation of a fluorescent-based system.

It would be desirable therefore to provide a safe, low-cost, fast and accurate method and apparatus for detecting the presence of biomolecules such as DNA, RNA, nucleic acid, proteins, antibodies and other ligands.

It would further be desirable to provide a method and apparatus for assaying the quantity of biomolecules at a given site.

### BRIEF DESCRIPTION OF THE DRAWINGS

FIG. 1 is a top view of a system for assaying the presence of a biomolecule in one embodiment of the present invention;

FIG. 2 is a side view of a given test site within the system for assaying the presence of a biomolecule shown in the embodiment of FIG. 1;

FIG. 3 shows three alternative configurations of the resonator of the system shown in the embodiment of FIG. 1;

FIG. 4 is a flow chart that illustrates one embodiment of a method of assaying the presence of a biomolecule according to the present invention;

FIG. 5 is a graph detailing the frequency shift in the high frequency response of an electrical resonator after an untagged solution of proteins was applied to the substrate; and

FIG. 6 is a graph detailing the frequency shift in the high frequency response of an electrical resonator after 0.1 and 1% magnetic solutions are applied to the substrate.

### DETAILED DESCRIPTION OF THE PRESENTLY PREFERRED EMBODIMENTS

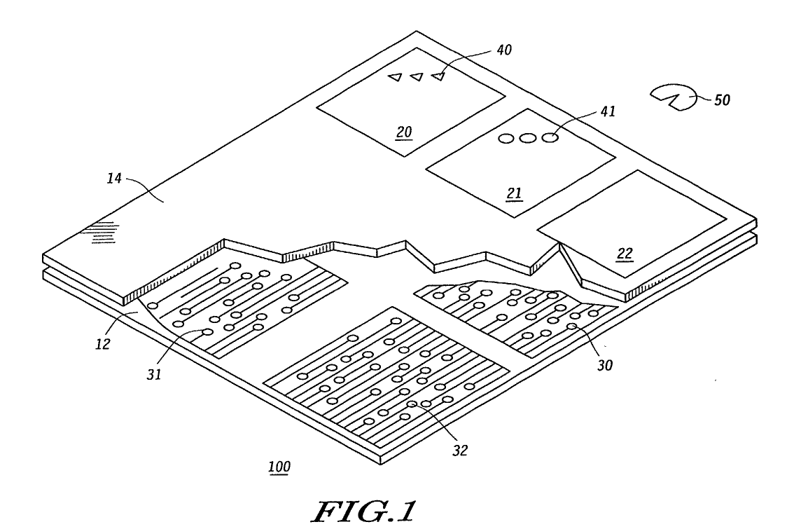

FIG. 1 illustrates one embodiment of a system 100 for assaying the presence of a biomolecule. The term “biomolecule” includes, for example, DNA, RNA, other nucleic acids, proteins, nucleotides, oligonucleotides, antibodies, antigens and cells. For example, system 100 could be used for cell detection, antibody detection or detection of any molecule.

The system 100 comprises an array of discrete test sites 20, 21, 22 disposed on a substrate 12. Substrate 12 may be a rigid substrate, such as glass, ceramic, silicon, nylon, or polypropylene. Substrate 12 may be formed using techniques, such as PWB technology or IC technology, for creating an electronic resonator surface (or resonant circuit). Such techniques are well known in the art. It is contemplated that test sites 20, 21, 22 may comprise indentations or wells in the substrate 12 or simply be discrete areas demarcated on the surface of substrate 12.

The substrate 12 may contain radio frequency passive components, such as, for example, inductors or resonators 30, 31, 32. In one embodiment, at least one such resonator 30 is preferably incorporated into the substrate 12. Alternatively, a resonator or radio frequency component may be distributed at each test site.

Resonators and radio frequency components are well known in the art. The RF resonator of the present invention may be, for example, a discrete or a distributed resonator.

In one embodiment of the invention, the resonator is a discrete RF resonator consisting of a parallel combination of an inductor “L” (in henries) and a capacitor “C” (in farads). In such a case, the resonance frequency “fc” (in hertz) for small electrical losses is given by fc=1/(2*pi*sqrt (L*C)). The resonator of the present invention may be a resonator having embedded discrete inductors and capacitors such as, for example, those described further in “Embedded Thin Film Resistors, Capacitors and Inductors in Flexible Polyimide Films”, from the Proceedings of 1996 Electronic Components and Technology Conference, herein incorporated by reference in its entirety. For example, the resonator may have embedded constructions in polyimide, which are similar to embedded constructions using other substrates.

Alternatively, the resonator may be a distributed resonator, such as, for example, those described in T. C. Edwards book FOUNDATIONS FOR MICROSTRIP CIRCUIT DESIGN, John Wiley & Sons, 1981, pp. 1-7, herein incorporated by reference in its entirety.

In one embodiment of the invention, the resonator may be a “far ground plane version” of an RFTV1 resonator, such as the resonator described, for example in “Using Resonators as Process Monitors to Ensure Consistent and Reliable RF Performance of HDI PCBs” from the Proceedings of IPC Expo 2001, herein incorporated by reference in its entirety. In one embodiment of the invention, the resonator may be, for example approximately 0.50×0.60 inches in size. This may be the lateral dimension of the resonator in terms of PWB area.

In some embodiments of the invention, substrate 12 may be a solid support as it well known in the art. Biomolecular receptors such as proteins, antibodies, and nucleic acids may be attached to a solid support using linker groups. Examples of such linker groups are thiols, amines, maleimides, sulfo NHS esters and photo reactive group such as acrylate, methacrylate, and dimethmaleimide. For example, bifunctional linker groups that will attach the receptor to the solid support are sold commercially by Pierce Endogen. In other instances, a polymer layer that contains a linker group can be coated on the solid support. A description of suitable polymer layers containing photosensitive linker groups is referenced in co-pending U.S. patent application Ser. No. 09/344,620, herein incorporated by reference in its entirety.

System 100 may also include a holding substance 14, which may be gel-like. This substance may be applied site by site, in one instance. Alternatively, the substance may be applied over the entire substrate. In one embodiment, the substance takes the form of a biogel, which is laid atop the substrate 12. A biogel may be any biocompatible polymer material that will bind or hold a biological probe. Examples of a biogel include polyacrylamide, polyethylene oxide, and polyvinyl alcohol. Other examples of a holding substance are described above in the discussion of suitable polymer layers containing linker groups. The holding substance preferably serves to hold test materials at the test sites.

As one illustration of the use of substrate 12 and substance 14, oligonucleotides may be modified with a reactive functional group (such as amine) and immobilized on the substrate using covalent linkers. Typically the substrate is coated with substance 14, which may be a coupling agent or thin polymer layer that will react with the functional group on the DNA.

A biological probe 40, 41 may then be applied to the test sites. Such a probe may be a ligand of the target molecule. In cases of known target molecules, the same probe may be used in every test site. In cases of unknown molecules, a different suspected ligand of the target molecule may be used at each test site. For example, in FIG. 1 one type of probe 40 is shown at site 20 and another type of probe 41 is shown at site 21. Thus if the probe 40 at site 20 is a ligand of the target molecule but the probe 41 at site 21 is not a ligand of the target molecule, the probe 40 will bind the target molecule at site 20 but no target molecule will be bound at site 21 (or at site 22, which does not carry any probes.)

A target molecule 50 is then applied to the test sites 20, 21, 22. In one embodiment, a solution containing target molecules 50 may be washed over the substrate. In one embodiment, the target molecule 50 may be applied to test sites on the substrate 12 without tagging or further such modification. In another embodiment, the target molecule 50 is tagged with a magnetic tag before being applied to the substrate. Such a magnetic tag may be, for example, ferrocene or magnetite. The target molecule 50 is allowed to interact with the probe on the substrate in a manner well known in the art, such as by binding.

The attachment of magnetic beads to biomolecules is well known to those skilled in the art. A very useful technique for attaching magnetic beads to biomolecules is to use streptavidin coated magnetic particles. Biomolecules (such as nucleic acids, proteins, sugars, enzymes, peptides) to be immobilized on the bead are first biotinylated and then bound to the streptavidin coated particles. Kits containing strepavidin coated magnetic particles and reagents for attaching proteins and nucleic acids are commercially available from companies such as Clontech and Genovision. A comprehensive list of enzymes that have been bound to magnetic particles is referenced in Scientific and Clinical Applications of Magnetic Carriers, Hafeli, Schutt, Teller, Zborowski, Plenum Press, New York. p 325, herein incorporated by reference in its entirety. Other methods of attaching magnetic particles to other molecules are described in U.S. Pat. No. 6,033,655; U.S. Pat. No. 6,204,033 and U.S. Pat. No. 6,200,755, herein incorporated by reference, each in its entirety.

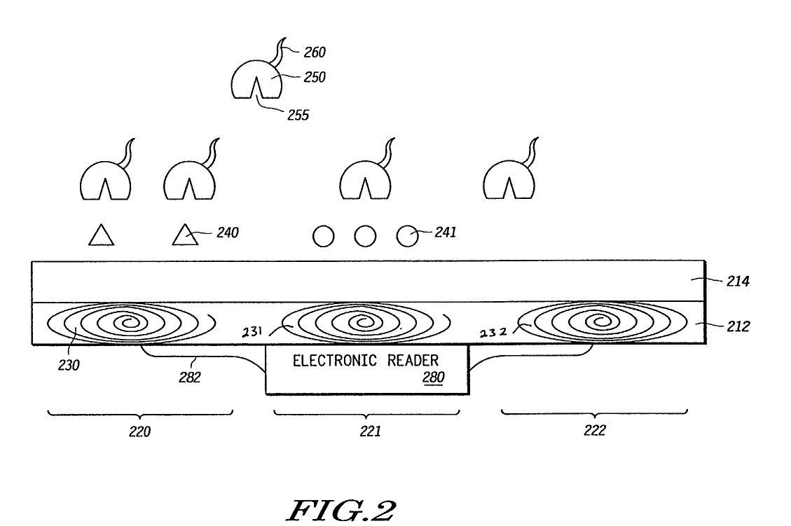

Referring now to FIG. 2, a cross-section of substrate 212 occupied by three test sites 220, 221, 222 is shown. It should be understood that many more such sites may be fabricated and tested on a single substrate 212. FIG. 2 also shows, for purposes of illustration, a substrate in contact with a holding substance 214.

Each test site contains a plurality of probes 240, 241 that are capable of interacting to known or unknown molecular structures or targets 250. The targets 250 could comprise, for example, biomolecules such as polynucleotides, DNA, RNA, cells, proteins, antibodies or anti-antibodies. In the case of an RNA or DNA sequence assay, the synthetic probes 240, 241 may comprise, for example, oligonucleotides. One example of a probe-target molecule interaction is binding between the probe and target molecule at a binding site. For example, target molecules 250 could bind to probes 240 at binding site 255.

All the probes 240 in a given test site 220 are preferably identical, for example, as shown at test site 220. However, the probes in respective test sites may differ within a single system 100. For example, as shown at test site 220, 221, two different types of probes are shown at 240 and 241 respectively. In one embodiment of the invention, the same probe 240 may be used in respective test sites at different concentrations to provide information regarding targets 250. Alternatively, probes 240 may differ for example in a known sequence for simultaneous detection of a plurality of different targets within a single system 100. Alternatively, the probes in respective test sites 220, 221, 222 may differ in a known sequence for simultaneous detection of a plurality of subsequences within a target molecule.

Probes 240, 241 may be selected according to the type of target that it is desired probes 240, 241 will bind. For example, if it is desired to bind a specific sequence of DNA, probes 240, 241 may comprise strands of DNA that are complementary to the desired DNA sequence. Oligonucleotides, single or double-stranded DNA or RNA, antibodies or antigen-antibodies complexes, tumor cells and other test probes known to those with skill in the art may be used. The probes may be attached to the test sites 220, 221, 222 by fixing them directly to substrate 212. Alternatively, as shown in FIG. 2, the probes may be fixed to a holding substrate 214.

At least one resonator 230 may be distributed within or proximate substrate 212. Preferably, a resonator 230 is distributed at each respective test site 220, 221, 222. As shown in FIG. 2, resonator 230 may have a spiral geometry.

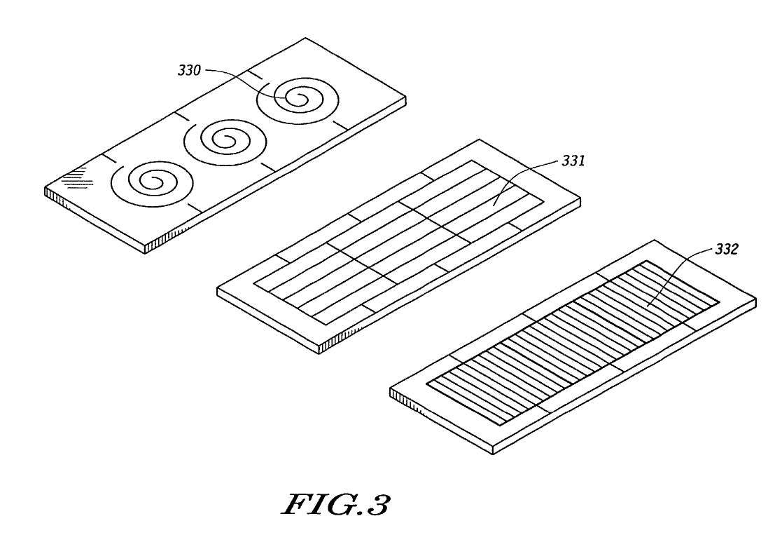

Referring to FIG. 3, alternative geometries of resonator 230 are shown. For example, a spiral geometry is shown at 330. However, other feasible geometries include structures made from any number of straight conductor segments. For example, a horizontal arrangement of straight conductor segments is shown at 331 and a vertical arrangement at 332.

Probes 240, 241 may be attached directly to resonator 230. Alternatively, resonator 230 may be distributed within substrate 212 and probes 240, 241 may be attached to the surface of substrate 212 proximate resonators 230. Alternatively, as shown in FIG. 2, probes 240 may be attached to holding substance 214, which is distributed on the surface of substrate 212. Holding substance 214 may also be distributed on the surface of resonators 230.

In one embodiment, resonator 230 is connected to an electronic reader 280. Electronic reader 280 is capable of analyzing the data generated by resonator 230. Thus, an electronic or magnetic property of the target 250 may be detected at test sites 220, 221, 222 by respective resonators 230, 231, 232. Conventional electrical equipment such as, for example, a contactless ID reader may serve as electronic reader 280. This reader may incorporate a frequency counting device or a spectrum analyzer as part of the method of detection. Reader 280 may also be for example an analyzer. Reader 280 may also include a component for recording the response of resonators 230, 231, 232. For example, in one embodiment the high frequency response of resonators 230 in the presence of targets 250 was recorded using HP 8719D network analyzers. As seen in FIG. 2, reader 280 may be located proximate a test site such as shown at site 221. Alternatively, reader 280 may be connected to a test site via an appropriate electrical connection 282 as shown at sites 220, 222.

Resonators 230 preferably detect the presence of targets 250 by a measured shift of the self-resonance frequency of the resonator 230. This measured shift is caused primarily by the modified effective capacitance of the resonator associated with the physical proximity of the targets 250 to the conductive traces of the resonator.

As seen in FIG. 2, magnetic tags 260 may be attached to targets 250 in order to increase the sensitivity of detection. In the case where magnetic tags are attached, the observed frequency shift is expected to be greater. This is due to the increased effective inductance of the resonator associated with the magnetic tags 260 in proximity to the resonators 230.

Test sites 220, 221, 222 may be etched on substrate 212 using print-etch techniques. Test sites 220,221, 212 may also comprise separate wells created within substrate 212. Substrate 212 may be, for example, organic or inorganic substrates, such as glass, polystyrenes, polyamides, polyamides, silicone dioxide, silicon, ceramic, and silicone nitride.

The solid support substrate 212 may be chosen in order to create a surface chemistry that is conducive to the formation of linkages between the substrate and the selected probes 240. For example, a glass support can be functionalized with an epoxide group by reacting it with an epoxy silane. The epoxide group on the support structure 212 reacts with a 5′-amino derivative oligonucleotides probe 240 to form a secondary amine linkage. Formation of this covalent linkage attaches the probes 240 to the support surface in a desired manner. Another example is a functionalized polystyrene surface that includes 5′ aldehyde or carboxylic acid derivatives coupled to a hydrazide-activated polystyrene. Additionally, the probes 240 may be attached to substrate 212 using holding substance 214. Also, the probes may be attached to substrate 212 using any appropriate attachment means, such as chemical linkages or physical linkages.

Alternatively, probes 240, 241 may also be attached directly to resonator 230. In this case, the resonators may be fabricated with materials capable of forming linkages with the probe 240, 241. Materials that may be incorporated into the surface of the resonators 230 or may be used to create the resonators to provide for direct attachment of probes 240, 241 include various conductive, semiconducting and dielectric materials, such as for example, gold, aluminum, copper, tin-indium-oxide, conducting inks and pastes, semiconducting organic and inorganic materials. Also, the biogel pad 214 itself may serve as a dielectric resonating structure.

Probes 240, 241 may be formed separately from substrate 212 and may then be inserted into each test site 220, 221, 222 by means well known in the art, such as, for example, via micropipettes. Alternatively, the probes 240, 241 may be synthesized within each test site at the time substrate 212 is formed.

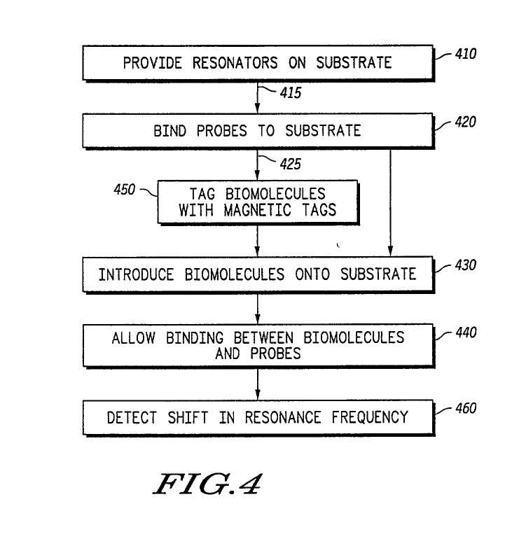

As FIG. 4 describes, the system 100 of the present invention may be used as a biosensor to sense the presence or absence of a given biomolecule. For purposes of illustration of a flow chart of one method for detecting and quantitating the presence of a biomolecule in accordance with the present invention is described below and shown in FIG. 4. A substrate containing integrated radio frequency components is provided at block 410. These components may be inductors or resonators as described above. A series of probes are bound to substrate 212 over the area of the radio frequency devices (at block 420). These probes are bound as described above. For example, these probes may be single DNA strands.

Preferably, a measurement is taken of the resonance frequency of the resonator(s) prior to application of the sample. This measurement will provide a value for comparison to the resonance frequency as measured after the samples are applied. Thus, a measurement may be taken of the resonance frequency at this time (at point 425). Alternatively, the measurement may be taken before the probes are applied (at point 415). In either case, the measurement will serve as a standard against which to ascertain whether or not a shift in frequency has occurred. Such a shift may be caused by biomolecules in the sample and may thus indicate their presence in the sample. In one embodiment, the carrier containing the biomolecules is a material or solution that will not cause this shift in frequency. Alternatively, the values for any resonance frequency shift that may be caused by the carrier may be determined for a “blank” sample (a sample of the carrier that contains no biomolecules). The experimental results may be appropriately corrected once these standard resonance values of the carrier are known.

A sample of targets 250 is then introduced onto the substrate (at block 430). For example, the DNA strands from a patient may be introduced onto the substrate by means well known in the art. The targets 250 are allowed to bind appropriately to the complementary probes (at block 440). For example, complementary strands of a patient's DNA may hybridize in a hybridization reaction to the single strand DNA probes on the substrate. Optionally, the targets may be tagged with magnetic microspheres or a similar paramagnetic moiety before being applied to the substrate (at block 450). For example, the DNA strands may be tagged with a paramagnetic moiety. Some examples of suitable tags include ferrocene and magnetite. Finally, the targets alter the high frequency response of the RF components. An electronic reader may be used to detect this alteration as a shift in the resonance frequency (at block 460).

The electronic reader may employ any of several methods to sense the presence or absence of a given biomolecule. These methods include, but are not limited to, a dissipation factor test, a transmission-loss detection test, a pulse and chirp method detection, an electrical resonance frequency test, and a test of electrical quality factor or Q. Methods of performing these electrical tests are well known to those skilled in the art.

FIG. 5 shows a graph detailing the frequency shift in the high frequency response of an electrical resonator system 100 after an untagged solution of proteins was applied to the substrate 12. In this example, 100 μg/per mil protein solution was used. The high frequency response for each of the electrical resonators was recorded for this sample after the biogel pad 14 was applied and after 40 μl of the protein solution was absorbed into the biogel pad. The resulting protein concentration was 0.67 nmoles. Up to a 14 MHz shift in resonance frequency was observed in the samples that contained untagged protein molecules (as seen at ΔF).

Thus the dotted line of FIG. 5 indicates a value that may have been taken at points 415 or 425 as described above, before the application of a sample containing untagged proteins. The solid line indicates the frequency of the resonators measured after the sample is added. A shift in frequency is indicated at ΔF. The shift may be caused by the untagged proteins and thus indicates the presence of these proteins in the sample.

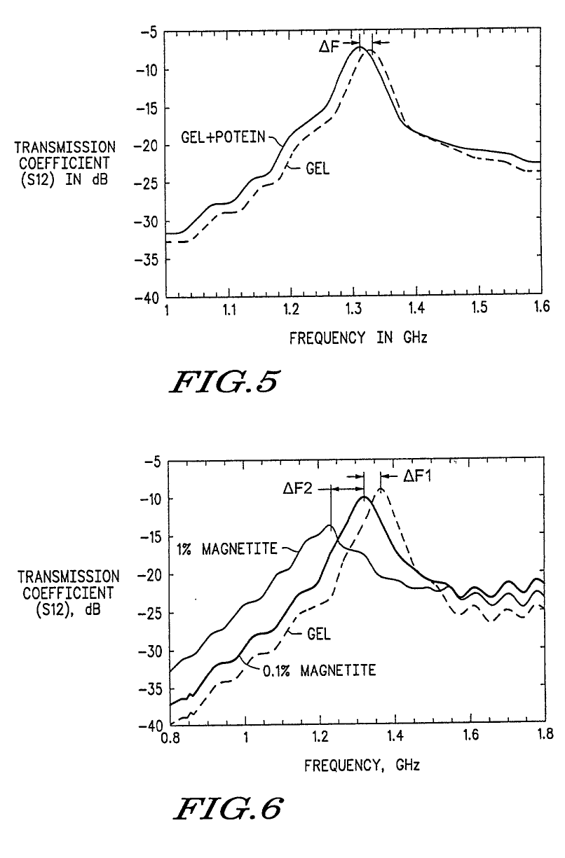

FIG. 6 shows the observed frequency shifts after two solutions of magnetite are applied to test sites within a system 100 as described above. The first solution contained 0.1% magnetite and the second contained 1.0% magnetite. Magnetite may serve as a suitable molecule for tagging biomolecules as described above.

The dotted line of FIG. 6 indicates a value that may have been taken at points 415 or 425 as described above, before the application of a sample containing magnetite. The dark solid line indicates the frequencies of the test sites measured after the 0.1% sample is added. A shift in frequency is indicated at ΔF1. The shift may be caused by the magnetite molecules and thus indicates the presence of these molecules in the sample. The light solid line indicates the frequencies of the test sites measured after the 1.0% sample is added. A shift in frequency greater than that seen with the 0.1% sample is indicated at ΔF2. The shift may be caused by the magnetite molecules and thus indicates the presence of these molecules in the sample.

Thus a sample containing biomolecules to which a solution of magnetite is added may provide a marked shift in resonance frequency as seen in FIG. 6 when the sample is applied to test sites of biomolecule detection system 100. Additionally, as can be seen from FIG. 6, the 1.0% magnetite sample gives a different set of resonance values than the 0.1% sample. Thus a sample containing more molecules may give a different set of values than a sample containing fewer molecules. Such resonance values may be correlated to number of molecules. System 100 may then be used to determine the amount or number of molecules present as indicated by resonance value.

### EXAMPLE 1

A substrate containing multiple surface resonator test sites of the above described geometry and specifications was fabricated by using standard printed circuit board print and etch technology, as is well known in the art. A polymer hydrogel coating containing biomolecular probe linker groups was draw coated onto the surface of 10 of the test sites to a thickness of 5 microns. The polymer coating was exposed to UV radiation of 1000 mJ/cm² at 365 nm to crosslink the polymer and render it insoluble to water solutions. 40 μL of a 100 μg/ml streptavidin protein was then washed over the surface of 5 of the resonators. The resonators were irradiated again with UV light at 365 nm (100 mJ/cm²) to photoactively couple the streptavidin to the probe linker groups in the polymer coating. The resonators were washed to remove excess material. The resulting protein concentration on the surface was approximately 0.67 nmoles per test site. The resonance frequency of the 5 test sites with the bound streptavidin were compared to 5 test sites with no streptavidin. The test sites containing streptavidin showed a 14 MHz frequency shift compared to the test sites with no streptavidin. This indicates that 0.67 nmoles per test site of a large protein such as streptavidin can be detected. Each test site is 1.94 cm² so this is a sensitivity of 0.35 nmoles/cm². In this case the frequency shift is due to the change in the capacitive properties of the test site due to the presence of streptavidin since the streptavidin does not contain a magnetic tag.

### EXAMPLE 2

A substrate containing multiple surface resonator test sites of the above described geometry and specifications was fabricated as is well known in the art. A polymer hydrogel coating containing biomolecular probe linker groups and 0.1% by weight colloidal magnetite dispersion (EMG 507 obtained from Ferrofluidics Corp., Nashua, N.H.) was draw coated onto the surface of 5 of the test sites to a thickness of 5 microns. A polymer hydrogel reference coating containing biomolecular linker groups and no magnetite particles was draw coated onto the surface of 5 of the test sites to a thickness of 5 microns. All the polymer coatings were exposed to UV radiation to render them insoluble as described above. The resonance frequency of the 5 test sites with the magnetite particles were compared to 5 test sites with no magnetite. The test sites containing magnetite showed a 32 MHz frequency shift compared to the test sites with no magnetite. This indicates a sensitivity to magnetite of that 10 nanograms per test site or 5 nanograms per cm².

Table 1 shows the observed frequency shifts and changes in the Q-factor of the electrical resonators under the influence of magnetic nano-particle that mimic magnetically tagged DNA molecules. A series of test sites containing different amounts of magnetite suspension on each test site was prepared. A 0.1% magnetite solution produced a 32 MHz frequency shift while a 1% magnetite solution produced a 127 MHz frequency shift. This shows that the frequency shift is quantitative and is increasing with increasing magnetite composition.

TABLE 1

| | Resonant | | |
|---|---|---|---|
| | Frequency, | | Frequency Shift, |
| Sample | Fc, GHz | Q | ΔF, MHz |
| BioGel pad | 1.3489 | 40.41 | N/A |
| 0.1% magnetite | 1.3172 | 24.34 | 32 |
| 1% magnetite | 1.2219 | 9.13 | 127 |

### EXAMPLE 3

A substrate containing multiple surface resonator test sites of the above described geometry and specifications was fabricated as is well known in the art. A polymer hydrogel coating containing biomolecular probe linker groups for streptavidin was coated onto the surface of 10 of the test sites to a thickness of 5 microns and UV irradiated as above. 5 of the test sites were washed with 0.1% by weight streptavidin coated magnetite particles and UV irradiated to attach the streptavidin to the polymer coating through the probe linker groups. The resonance frequency of the 5 test sites with the magnetite particles were compared to 5 test sites with no magnetite. The test sites containing magnetite showed a 32 MHz frequency shift compared to the test sites with no magnetite. This indicates a sensitivity to streptavidin magnetite of 10 nanograms per test site or 2.5 nmoles of streptavidin per test site.

Other sample characteristics may therefore be extracted from this method beyond the simple detection that DNA is present and has bound a particular complementary oligonucleotide.

The high sensitivity and quantitative results of this method allow formulation of industry-wide standards based on acceptable sensitivity levels, repeatability of measurements, type of information obtained, etc.

One embodiment of the present invention may provide biomolecule detection system 100 in a kit form. Biological kits may provide the necessary tools and reagents for an assay in a standardized, convenient form. Such a kit may include a substrate with a resonator (which may be attached to or detachable from the resonator). The kit may further include reagents such as a holding substance for adding to the substrate or probes for specific or well-known targets. The kit may also include reagents for tagging the unknown or target molecules, such as solutions of ferrocene.

It should be appreciated that the embodiments described above are to be considered in all respects only illustrative and not restrictive. The scope of the invention is indicated by the following claims rather than by the foregoing description. All changes that come within the meaning and range of equivalents are to be embraced within their scope.

We claim:

1. A method of detecting a biomolecule, comprising the steps of:
providing a substrate, the substrate having at least one biodetection site including an electrical resonator therein;
applying at least one biochemical probe to the biodetection site;
interacting the biomolecule with the biochemical probe at the biodetection site; and
detecting the biomolecule at the biodetection site.

2. The method of claim 1 further comprising:
applying a magnetic tag to the biomolecule.

3. The method of claim 1 further comprising:
detecting magnetic properties of the biodetection site with the resonator.

4. The method of claim 3 wherein a value of the magnetic properties of the biodetection site corresponds to a number of biomolecules at the biodetection site.

5. The method of claim 1 further comprising the step of:
applying a holding substance to the biodetection site.

6. The method of claim 1 wherein the substrate comprises an organic substrate with embedded resonance frequency structures.

7. The method of claim 1 wherein the biochemical probe is an oligonucleotide.

8. The method of claim 1 wherein the magnetic tag is ferrocene.

9. The method of claim 1 further comprising:
measuring the magnetic properties of the biodetection site before applying the biomolecule.

10. The method of claim 9 further comprising determining an alteration in magnetic properties of the biodetection site.

11. The method of claim 9 wherein a value of the magnetic properties of the biodetection site corresponds to a number of biomolecules at the biodetection site, further comprising the steps of:
correlating a value of the alteration in magnetic properties with a number of biomolecules; and
determining the number of biomolecules at the biodetection site based on the value of the alteration.

12. A method for detecting a biomolecule comprising the steps of:
reacting a biomolecule with a magnetic tag;
contacting the tagged biomolecule to a biodetection site on a resonator, the site including a solution of at least one probe;
coupling the biomolecule to the probe; and
detecting the number of biomolecules at the site.

13. A biosensor for assaying biomolecules, comprising:
a substrate having at least one biodetection site; and
an electrical resonator proximate the biodetection site to allow measurement of magnetic properties at the biodetection site.

14. The biosensor of claim 13 wherein the biodetection site includes a holding substance.

15. The biosensor of claim 13 wherein at least one biochemical probe is present at the biodetection site.

16. The biosensor of claim 13 wherein the biodetection site is adapted to receive at least one biomolecule, further comprising:
at least one magnetic tag operatively adapted to be applied to the biomolecule.

17. The biosensor of claim 13 wherein the electrical resonator measures a value of magnetic properties, the value corresponding to a number of biomolecules at the biodetection site.

18. The biosensor of claim 13 wherein the electrical resonator has a spiral geometry.

19. The biosensor of claim 13 further comprising:
a measuring component for measuring the magnetic properties in communication with the biodetection site.

20. The biosensor of claim 19 further comprising:
at least one quantitating element for correlating an amount of biomolecules at the biodetection site to the magnetic properties of the biodetection site.

21. A kit for assaying target biomolecules, comprising:
a sample plate including a plurality of biodetection sites;
a holding gel adapted to coat the biodetection sites; and
an electrical resonator operatively attached to the sample plate to allow measurement of magnetic properties of the biodetection sites.

22. The kit of claim 21 further comprising:
a solution of at least one biochemical probe adapted to be applied to the biodetection sites.

23. The kit of claim 21 further comprising:
a solution of at least one magnetic tag adapted to be applied to the target biomolecules.
